(19) United States
(12) Patent Application Publication
(10) Pub. No.: US 2010/0317631 A1
(43) Pub. Date: Dec. 16, 2010

(54) Gel Composition and Transcrotal Application of a Composition for the Treatment of Hypogonadism

(75) Inventors: Bernd SCHULZE, Bielefeld (DE); Eberhard Nieschlag, Munster (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Dr. August Wolff GmbH & Co. Arzneimittel, Bielefeld (DE)

(21) Appl. No.: 12/795,348

(22) Filed: Jun. 7, 2010

Related U.S. Application Data
(63) Continuation of application No. 10/451,482, filed on Sep. 23, 2003, now abandoned.

(30) Foreign Application Priority Data
Dec. 22, 2000 (DE) 10064205.5

(51) Int. Cl. A61K 31/569; A61K 31/56; A61P 25/24; A61P 7/06; A61P 15/10; A61P 19/10

(52) U.S. Cl. 514/170; 514/169; 514/179

(57) ABSTRACT

The present invention relates to a gel composition on alcoholic basis, comprising at least one androgenic steroid and at least one C₃-C₄ diol as enhancer, as well as the use of a composition comprising at least one androgenic steroid on the scrotum for the treatment and/or prophylaxis of hypogonadism.

The present invention relates to a gel composition as well as to the use of a composition for the treatment and/or prophylaxis of hypogonadism.

Masculine sex hormones, the androgens, are responsible for the development of the masculine sex characteristics. Furthermore, they are required for reproduction. The main element of the androgens is testosterone, which is imperative for the development and the function of the internal and external masculine sex organs, which has a supportive influence regarding muscle growth, which determines the distribution and the density of hair growth, which has a positive influence with respect to the production of erythrocytes and with respect to the distribution of erythropoietin and the cognitive functions. A shortage of testosterone (hypogonadism) may be classified into two principle forms, which are designated primary and secondary hypogonadism. The lack of testosterone production or a decreased testosterone production within the body, originating from a malfunction of the testicles, which is the main synthesis location of testosterone, is designated primary hypogonadism. Is the main reason for the diseases, however, a malfunction of the hypothalamus or the hypophyse the disease is named secondary hypogonadism. One indication for the therapy of both forms of hypogonadism is the finding that the testosterone concentration in serum decreases below 12 nmol/l during the morning hours, occurring for example in connection with symptoms of androgen shortage, such as for example diseases based on testosterone shortage such as osteoporosis, muscle atrophy, senescence outfall symptoms, the decrease of libido and potency, depression and anaemia. The treatment usually is a substitution therapy which effectively can be measured directly based on the testosterone concentration in serum. The aim of the testosterones substitution is to increase the testosterone concentration in serum to the normal value.

Chemically testosterone, 17-β-hydroxy-androst-4-ene-3-one, is derived from the main structure of all androgens, androstan. The specific biological activity of testosterone is based on the keto group in position 3, the double bond in position 4 and the hydroxy group in position 17 of the androstan main structure. In order to use testosterone during therapy it can be used as such or in chemically modified form, such as for example by introducing an ester group at the position 17. In particular, for the therapy using a testosterone preparation the type of application is important. Implants comprising testosterone, testosterone preparations for oral use, testosterone preparations for intramuscular application and transdermal testosterone compositions are used in the art. A survey with respect to the question of therapy using testosterone has been published by E. Nieschlag and N. M. Behre in Andrologie, Grundlagen and Klinik der Reproduktiven Gesundheit des Mannes, pages 315 to 329, Springer Verlag, 1996.

Testosterone implants consist of pure testosterone, cast to a cylindrical form having a length of 12 mm and a diameter of 4.5 mm. One implant contains 200 mg. If 3 to 6 implants are applied, slowly decreasing concentrations of testosterone in serum within the normal range are obtained for 4 to 6 months. The surgical activity necessary for implantation, the extrusion of the implants which can be observed with 5% of the patients and infections and occasional bleeding represent the severe drawbacks of testosterone implants. Implants are commercially available only in the United Kingdom and Australia.

Orally administrated free testosterone is metabolised in the liver completely, so that the target organs cannot be reached. During oral therapy, therefore preparations comprising chemically modified testosterone are used, such as testosterone being converted into an ester at the position 17β with undecanoic acid or testosterone being methylated at the position 17α.

If natural testosterone is administered intra muscular, the half time value is very short. In order to increase the duration of activity testosterone has been converted into an ester using aliphatic side chains at the position 17. The most common substitution therapy comprises the intra muscular administration of testosterone enanthat, which shows a terminal half-time value of 4.5 days. The standard dosage is 250 mg testosterone enanthat. With this therapy high testosterone concentrations in serum of up to 50 nmol/l are achieved very quickly, which gradually decrease and usually pass the lower limit of normal range after the 12^{th} day. A repetition of injections, necessary for the substitution therapy, gives rise to a so-called “saw-tooth” profile comprising, depending from the interval of the injections, different phases having supra physiological values, physiological values and infra physiological values. While this form of substitution is sufficient in order to maintain the biological activity of the testosterone, the patient usually regards these strong differences as detrimental, since the overall well being, the mood and the sexual activity follow this “saw-tooth” profile.

The development of transdermal testosterone preparations has enriched the therapy with respect to diseases associated with testosterone shortage with one further interesting alternative. The transdermal application has, particularly in the endocrinological area, achieved some importance with respect to the administration of oestrogen as regards the menopause treatment. The development of transdermal systems with respect to the substitution therapy of hypogonadism, however, has encountered problems, since the treatment of masculine patients requires the application of dosages within the range of the daily production of testosterone, i.e. 6 mg per day. The corresponding value for the administration of oestradiolum for feminine patients however lies within the microgram region.

Different parts of the skin do show differing abilities for resorption. In view of the fact that scrotal skin serves as temperature regulating means so that this skin is heavily supplied with blood even in the upper layers of the epithel, this skin has a resorption ability which is very high (compared with the skin of the lower arm by a factor of 40). This has lead to the development of a trans scrotal application system. This system comprises a plaster with testosterone (Testoderm®) comprising 40 to 60 cm² membranes consisting of polymer, loaded with 10 to 15 mg of pure natural testosterone. If this membrane is applied onto the scrotum sufficiently high doses of testosterone are transferred in order to guarantee a physiological testosterone value in serum for one day. If the membranes are applied during the morning hours, even the physiological day rhythm of the testosterone may be imitated. The application of a plaster however requires an appropriate preparation. In order to guarantee the intimate skin contact the scrotum must be free of hair by shaving or by using scissors. Since the plaster does not contain any adhesive, the plaster must be, in order to achieve a sufficient fixation on the scrotum, warmed up using a hair dryer. Upon removal, the plaster must be warmed again in order to release it from the scrotum. The plaster comprising testosterone on the scrotum however is regarded by many patients as being uncomfortable. Irritation of the skin and itching are further drawbacks of this type of testosterone therapy.

In addition, transdermal testosterone plasters have been developed which are applied until non-scrotal skin (for example stomach, forearm, breast or back) (Androderm®). In order to transfer the required amount of testosterone through the skin these systems do comprise a resorption accelerator (enhancer). This gives rise to a high percentage of irritations of the skin, which may for example give rise to severe skin diseases. These plasters furthermore can be easily detected and do not enable a flexible dosage.

In addition, transdermal applications of testosterone in gel compositions are known. In France the transdermal preparation Andractim® is known, which comprises 5α-dihydrotestosterone with a concentration of 2.5% within a hydro alcoholic gel. If this gel is applied onto sufficiently large areas of the skin (arms, shoulders, breast, stomach or thighs), the dihydrotestosterone penetrates into the skin and it is possible to detect supraphysiological dihydrotestosterone concentration in serum.

A further dermal therapy system on the basis of a hydro alcoholic gel comprising 1 wt % testosterone has received approval of the FDA recently. This system is distributed by the company named Unimed Pharmaceuticals, Inc under the trade name Androgel®. This product is applied to the arms, shoulders and/or the front side of the main body. However, the application of a gel onto a large area of the skin seems to be not practical since, by means of skin contact or contact with clothes, an unwanted testosterone transfer to a partner may occur, which may give rise to undesired viralisation. This therapy therefore requires the removal of the applied gel prior to sexual or social contact with persons of the other sex.

It is therefore the object of the present invention to solve the above-mentioned problems or at least to alleviate those problems and to provide a medicament which can be used for the treatment and/or prophylaxis of hypogonadism as well as the symptoms associated therewith.

This object is solved in accordance with the present invention by means of a gel composition on the basis of alcohols, comprising at least one androgenic steroid and at least one C₃ to C₄ diol as resorption increasing agent.

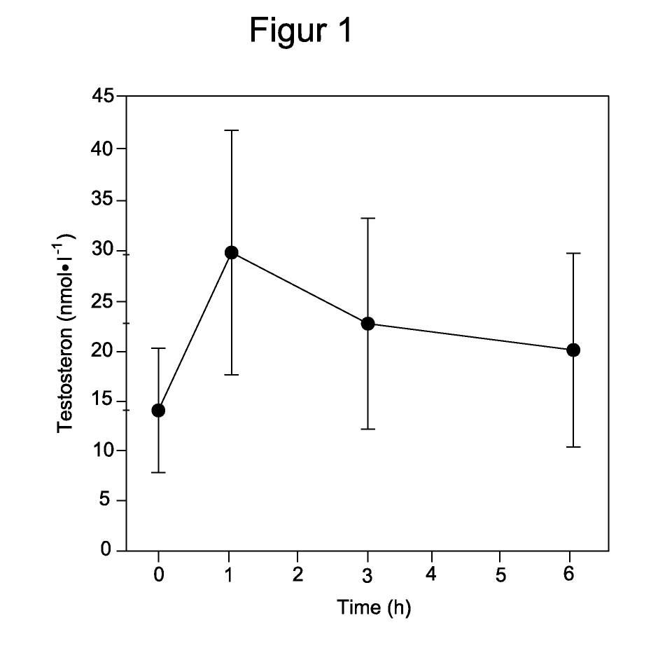

FIG. 1 shows the average concentration of testosterone in serum 0 to 6 hours after the scrotal application of 1 g of a testosterone gel.

Surprisingly it was found that the gel composition in accordance with the present invention enables an improved penetration of the androgenic steroid through the skin into the blood cycle. In particular it has been found that, contrary to the testosterone compositions, such as the testosterone gels known in the prior art, the gel composition in accordance with the present invention may be washed away about 10 minutes after application, without leading to a suppression of the penetration process. To the contrary, an increase of the penetration process can be detected. It was found therefore that the mechanical factor of washing away the gel composition apparently provides an additional penetration accelerating activity. In this connection, it could be proved that, after application of the gel composition in accordance with the present invention and subsequent washing away after 10 minutes, a remarkable increase of the testosterone concentration in blood could be detected, which lead to a normalisation of the hypogonadel values. Since the gel composition in accordance with the present invention provides the possibility to remove the gel about 10 minutes after application by washing away the gel it can be secured that a contamination of persons of the other sex or of children during sexual and social contact can be prevented. In addition, the success of therapy for the person who has to use the gel composition in accordance with the present invention is not endangered.

The gel composition in accordance with the present invention therefore enables an improved resorption of the active ingredient within a short time. This phenomenon is not decreased if the gel composition in accordance with the present invention is washed away after a relatively short period of time. To the contrary, this phenomenon is increased. Within a short time a sufficient amount of active ingredient enters into the blood stream so that an effective increase of hormone concentration can be reached, while at the same time a contamination of persons of the other sex and children is prevented.

The compounds used in accordance with the present invention and named androgenic steroid comprise natural and synthetic androgenic steroids, which give rise to a reduction of the symptoms of testosterone shortage in men. Preferably the androgenic steroids in accordance with the present invention are selected among the group consisting of testosterone, testosterone esters, methyl testosterone, methyl testosterone esters, androtestone dione, andrenosteron, dehydroepiandrosterone, fluoxymesterone, methandrostenolone, 17α-methylnortestosterone, norethandrolone, dihydrotestosterone, oxymetholone, stanozolol, ethylestrenol, oxandrolone, bolasterone and methyl testosterone. Among this group testosterone, testosterone esters, methyl testosterone, dihydrotestosterone or mixtures thereof are preferred. Testosterone esters may be proprionates, phenylacetates, enanthates, undecanoates, cypionates or buciclates. In particular testosterone is preferred.

The steroid content of the gel composition in accordance with the present invention preferably is 0.01 to 10 wt %, more preferably 0.1 to 4 wt % and more preferably 2 to 7 wt % and most preferably 2 to 3 wt %, based on the weight of the gel composition.

The C₃ to C₄ diol usually is propylene glycol, a butylene glycol, including 1,3-dihydroxybutane, 2,3-dihydroxybutane and 1,4-dihydroxybutane as well as mixtures of these diols. Preferably propylene glycol is used, alone or together with at least one butylene glycol.

The gel composition in accordance with the present invention comprises the at least one C₃ to C₄ diol in an amount of from 0.1 to 20 wt %, preferably 0.5 to 10 wt %, more preferably 1 to 10 wt % and most preferably 3 to 8 wt %, based on the weight of the gel composition.

The gel composition in accordance with the present invention is formulated on an alcoholic basis. Examples of the alcohols comprise C₂ to C₇ alcohols, preferably C₂ to C₄ alcohols, in particular ethanol and propanol.

The gel composition in accordance with the present invention is formulated usually in such a manner that the alcohol makes up for at least a major portion. In accordance therewith, the gel composition in accordance with the present invention comprises alcohol in an amount from 30-90 weight % more preferable 40-70 weight % and most preferably 50-60 weight %, based on the weight of the gel composition.

It is preferred that the gel composition in accordance with the present invention furthermore comprises a pH stabilizer in order to enable a homogeneous composition of the gel. In this respect, the usual pH-stabilizers may be used. Usual are weak acids, weak bases, weak acidic buffers and/or weak basic buffers. In particularly suitable are weak basic or weak acidic buffers. Particularly suitable as buffer is Tris-Buffer (α,α,α-Tris(Hydroxymethyl)-methylamine, Tromethamol) or salts thereof.

The pH-stabilizer is usually present in an amount in order to guarantee the stability of the gel composition in accordance with the present invention. Usual are amounts of 0 to 2 weight % preferably 0.01 to 1 weight %, more preferably 0.05 to 0.5 weight %, based on the weight of the gel composition.

As further component, the gel composition in accordance with the present invention, furthermore may comprise a polymer on the basis of poly(meth)acrylic acid, preferably in order to improve the viscosity or the structure of the gel composition in accordance with the present invention. Preferred polymers are selected from the group of carbomeres, which have an acrylic acid backbone and comprise minor amounts of polyalkenylpolyether as cross-linking agents, and in addition optional comonomers such as C₁₀ to C₃₀ alkyacrylates. Usual carbomeres are commonly known and comprise, e.g., carbopol 980, carbopol 941, carbopol 940, carbopol 934, carbopol ultrez U 10 as well as carbopol ETD-2020, which may be used singly or in mixture.

The content of polymer amounts usually to 0-10 weight % preferably 0.1 to 6 weight % more preferably 0.5 to 4 weight % based on the weight of the composition.

The preparation of the gel composition in accordance with the present invention usually is carried out by mixing at least one androgenic steroid and at least one C₃ to C₄ diol optionally together with the additional components and/or carriers in order to bring the formulation to a desired form. The additional components and carriers originate from a group of carriers, preservatives and other usual additives.

The gel composition in accordance with the present invention may be applied onto specific parts of the body, e.g., the stomach, arms, including forearm and upper arm, legs, including upper and lower leg, breast, back and scrotum, in order to achieve treatment and/or prophylaxis of hypogonadism. The diseases associated with testosterone shortage in the connection of hypogonadism comprise e.g., osteoporosis, muscle atrophy, senescence outfall symptoms, loss of libido and potency, depression and anaemia.

The gel composition in accordance with the present invention may comprise usual carriers, excipients as well as further active ingredients. Preferred excipients originate from the group of anti-oxidants, preservatives, stabilizers, solution enhancers, vitamins, colouring agents and odour-improving agents.

The gel composition in accordance with the present invention may, in addition to one or more androgenic steroids, comprise usual carriers, such as fats of animal or vegetable origin, waxes, paraffins, starches, traganth, cellulose derivatives, polyethylene glycols, ethanol, propanol, polyacrylic acid, silicones, bentonite, silicic acid, talc and zinc oxide or mixtures of these carriers.

Preferably, the gel composition in accordance with the present invention comprises, in addition to the C₃ to C₄ diol, at least one further resorption improver (enhancer). Examples of suitable enhancers are fatty acids, fatty acid esters, fatty alcohols, sorbat ester and salts thereof, glycerine esters of fatty acids, fatty acid esters of α-hydroxy acids and mixtures of these compounds. The enhancers are contained preferably in an amount of 0.01 to 30 weight % based on the composition. Even some solvents may function as enhancers. As examples, C₂ to C₇ alcohols, ethoxydiglycol, DMSO, SMF, DMA, 1-n-Dodecylcycloazacycloheptan-2-on, NMP and N-(2-hydroxyethyl)pyrrolidon can be named.

Additional additives, such as thickeners, gums and agents that increase the solubility of androgenic steroids, may preferably be present in the gel composition in accordance with the present invention. Preferred are particular compounds which increase the solubility of androgenic steroids in hydrophilic compositions, particularly cyclodextrines, such as α, β- and/or γ-cyclodextrine.

The amount of the gel composition in accordance with the present invention which is applied to the specific part of the body, preferably amounts to 0.2 to 20.0 grams, in particular 1.0 to 10.0 grams, and most preferably 1.0 to 7.0 grams.

The amount of steroid per dose to be applied accordingly amounts of 40 to 400 mg, preferably 50 to 300 mg, more preferably 70 to 250 mg and most preferably 100 to 250 mg.

The application of the gel composition in accordance with the present invention preferably occurs by using a tube, a soft-gelatine capsule or a dosage apparatus with or without aerosol.

In a further embodiment, the present invention relates to the use of a composition comprising at least one androgenic steroid on the scrotum for the treatment and/or prophylaxis of hypogonadism.

The present invention furthermore relates to the use of a composition comprising at least one androgenic steroid and at least one C₃ to C₄ diol for the treatment and/or prophylaxis of hypogonadism.

Surprisingly it was found that, during the usage in accordance with the present invention, which comprises the application of the composition onto the scrotum, even the application of rather minor amounts is sufficient in order to achieve a permanent testosterone concentration in blood, imitating the natural testosterone concentration in serum. This effect cannot be explained fully based on the resorption capability of the skin of the scrotum only. Contrary to the usual transdermal therapy systems, the massage of the scrotum, which occurs inevitably when using the composition, achieves this improvement. Surprisingly this effect, which may be described as a synergistic effect, even extends to exogenic testosterone applications.

The concentration of androgenic steroids in the compositions used in accordance with the present invention, required in order to achieve a normal testosterone concentration in the serum, is therefore, compared with usual transdermal application systems very low.

In view of the problems associated with the trans-scrotal application of testosterone plasters, the application of a gel onto the very sensitive skin of the scrotum should give rise to massive irritations of the skin. Surprisingly it was found that the drawbacks associated with the prior art do not occur when using the composition in accordance with the present invention.

The usage in accordance with the present invention, comprising the application of the composition onto the scrotum, prevents, contrary to the transdermal application onto the upper arm, the upper legs, the stomach, and the breast, the undesired transfer of androgenic steroids onto a partner during intense contact, e.g., during sexual intercourse. Furthermore, this undesired transfer can be completely excluded during usual social contacts with children or women.

The compounds named androgenic steroids, used in accordance with the present invention, comprise natural and synthetic androgenic steroids, which may give rise to a reduction of the symptoms of testosterone shortage in men. Preferably the androgenic steroids to be used in accordance with the present invention are selected from the group consisting of testosterone, testosterone esters, methyl testosterone, methyl testosterone esters, androstendion, andrenosterone, dehydroepiandrosterone, fluoxymesterone, methandrostenolone, 17α-methylnortestosterone, norethandrolone, dihydrotestosterone, oxymetholone, stanozolol, ethylestrenol, oxandrolone, bolasterone, and mesterolone. From this group preferred are: testosterone, testosterone esters, methyl testosterone, dihydrotestosterone, and mixtures thereof. Testosterone esters can be propionates, phenyl acetates, enanthates, undecanoates, cypionates or duciclates. Testosterone is especially preferred.

The preparation of the composition used in accordance with the present invention occurs usually by mixing at least one androgenic steroid, optionally together with excipients and/or carriers in order to bring same into a suitable dosage form. The excipients and carriers are selected from the group of carriers, preservatives and other usual excipients.

The compositions are used in accordance with the present invention, in particular on the scrotum for the treatment and/or prophylaxis of hypogonadism. The diseases associated with the testosterone shortage in connection with hypogonadism comprise e.g., osteoporosis, muscle atrophy, senescence outfall symptoms, loss of libido and potency, depression and anaemia.

The formulation is preferred for topical application. As application forms, solutions, suspensions, emulsions, pastes, ointments, gels, creams, lotions and oils may be named. Preferred the composition in accordance with the present invention is present in the form of a gel.

The composition may further comprise usual carriers, excipients and optional further active ingredients. Preferred excipients are selected from the group of preservatives, antioxidants, stabilizers, solution enhancing agents, vitamins, colouring agents and odour-improving agents.

Ointments, pastes, creams and gels may comprise, in addition to the at least one or more androgenic steroids, the usual carriers, e.g., fats of animal or vegetable origin, waxes, paraffins, starches, traganth, cellulose derivatives, polyethylene glycols, ethanol, propanol, polyacrylic acid, silicones, bentonite, silicic acid, talc and zinc oxide or mixtures of these carriers.

Solutions and emulsions may comprise, in addition to the at least one or more androgenic steroids, the usual carriers, such as solvents, solvent enhancing agents, and emulators, e.g., water, ethanol, isopropanol, ethylcarbonat, ethylacetat, benzyl alcohols, benzyl benzoate, propylgenglycol, 1-3-butyglycol, oils, especially cotton-seed oil, peanut oil, corn oil, olive oil, caster oil and sesame oil, glycerine fatty acids esters, polyethylene glycols, fatty acid esters sorbitans or mixtures of these components.

Suspensions may comprise in addition to the at least one or more androgenic steroids, the usual carriers, such as liquid dilutants, e.g. water, ethanol or propylgenglycol, suspending agents, e.g., ethoxydated isostearylalcohols, polyoxyethylene sorbitan esters and polyoxyethylene sorbitan esters, microcrystalline cellulose, aluminiummetahydroxid, betonite, agar-agar and traganth or mixtures of these components.

Oils may comprise, in addition to the at least one or more androgenic steroids, the usual carriers, such as synthentic oils, e.g., fatty acids, fatty alcohols, silicone oils, natural oils such as vegetable oils and oily plant extracts, paraffin oils, lanolin oils or mixtures of these components.

Preferably, the composition used in accordance with the present invention comprises a resorption-improving agent (enhancer). Examples of suitable enhancers include fatty acids, fatty acid esters, fatty alcohols, sorbat ester and salts thereof, glycerine esters of fatty acids, fatty acid esters of α-hydroxy acids and mixtures thereof. Enhancers are present preferably in an amount of 0.01 to 30 weight % based on the composition. Even specific solvents may function as enhancers. In this connection, C₂ to C₇ alcohols, C₃ or C₄ diols, ethoxydiglycol, DMSO, SMF, DMA, 1-n-dodecylcycloazacycloheptan-2-on, NMP and N-(2-hydroxyethyl)pyrrolidon may be named.

Further additives, such as thickeners, gums and agents which improve the solubility of androgenic steroids, may preferably be contained in the gel composition in accordance with the present invention. Especially preferred are compounds which increase the solubility of androgenic steroids in hydrophilic compositions, particularly cyclodextrines, such as α-, β- and/or γ-cyclodextrine.

The steroid content of the composition used in accordance with the present invention preferably amounts from 0.01 to 10 weight %, more preferably 0.1 to 4 weight %, even more preferably 2 to 7 weight % and most preferably 2 to 3 weight % based on the weight of the composition.

The amount of the composition used in accordance with the present invention on the scrotum amounts to 0.2 to 20.0 grams, more preferably 1.0 to 10.0 gram and most preferably 1.0 to 7.0 grams.

The application of the composition used in accordance with the present invention onto the scrotum preferably occurs by using a tube, a soft-gelatine capsule or a dosage apparatus with or without aerosol.

The following examples illustrate the invention. All compounds or components which may be used in the formulations are either known and may be purchased or may be synthesized using known methods.

### EXAMPLE 1

In Table 1 formulations for gels (G-I, G-II, and G-III) as well as solutions (LI, L-II, and LIII) are represented in parts by weight, which may be used in accordance with the present invention.

TABLE 1

| | G-I | G-II | G-III | L-I | L-II | L-III |
|---|---|---|---|---|---|---|
| Testosterone | 2.5 | 2.5 | 1.0 | 2.5 | 2.5 | 1.0 |
| α-bisabolol | 1.0 | — | — | 1.0 | — | — |
| Lecithin | 1.5 | — | — | 1.5 | — | — |
| Ethanol | 51.5 | 56.0 | 56.0 | 51.5 | 56.0 | 56.0 |
| (96%) | | | | | | |
| Propylene | 5.0 | 5.0 | 5.0 | 5.0 | 5.0 | 5.0 |
| glycol | | | | | | |
| Carbopol | 1.0 | 1.0 | 1.0 | — | — | — |
| 980 | | | | | | |
| Trometha | 0.14 | 0.14 | 0.14 | — | — | — |
| mol | | | | | | |
| water | 37.36 | 35.36 | 36.86 | 38.5 | 36.5 | 38.0 |

For the evaluation of percutaneous resorption of testosterone during scrotal application the following evaluation was carried out:

Six test persons were applied with 1 gram gel (G-II) at 8:00 am using rubber gloves on the scrotal region. The test persons were instructed not to wash the scrotum prior to the lapse of six hours after application and to avoid skin contact of treated areas with other persons. In the time between 7:30 and 8:00 am, prior to the application of the gel, blood samples were taken from each test person in order to determine the starting value for the hormone. Subsequent to application one hour, three hours and six hours (9:00 am, 11:00 am and 2:00 pm) blood samples were taken in order to determine the serum values for testosterone.

The results of the hormone analysis are depicted in FIG. 1. The average testosterone starting value was 14.0 nmol/l. Average testosterone after one, three and six hours were 29.5, 22.7, and 20.0 nmol/l respectively. The vertical lines in FIG. 1 show the standard deviation of the determined values due to the different increase and decrease of testosterone concentration in the individual test persons.

The transscrotal application makes it possible to reduce considerably the amount of gel and the dosis of testosterone respectively as regards the prior art, while simultaneously avoiding the problems associated with the prior art, described above, yet still achieving the necessary normalization of the concentration in order to treat hypogonadism.

### EXAMPLE 2

For comparative purposes, a further evaluation was carried out with six further test persons which applied 5 gram gel over the breast, stomach or upper arm (compare with one gram gel onto the scrotum). The average testosterone values in serum as determined did not show any deviation from the values as depicted in FIG. 1.

### EXAMPLE 3

Gel G-II, as prepared in Example 1, having a testosterone concentration of 2.5 weight %, was used in a pilot study with respect to pharmacokinetic and pharmacodynamic properties. This study had the aim to evaluate the influence of the testosterone gel with respect to the concentration, that had been measured over 24 hours, over a period of ten days of application. In addition, the possibility to wash away the gel after 10 minutes and the amount of testosterone transferred into the blood after skin contact were evaluated.

Fourteen male test persons applied daily 5 grams of the testosterone gel onto the upper body. Of these test persons, 7 washed away the gel after 10 minutes of application.

The target parameter for the determination of the testosterone resorption were serum concentrations over 24 hours and 10 days. The criteria as employed were area below the concentration-time-curve (AUC₀₋₂₄) and the maximum serum concentration (Cₘₐₓ) as well as the duration of the increased serum concentration (t) due to the application of the gel.

The testosterone gel as used enables an adequate testosterone substitution. A supraphysiological increase of estradiol and DHT was not observed. Accumulation of the testosterone concentration in the serum was found from the first to the tenth day (δCmax day 1: 10.0 nmol/l±8.8 MW±Sdev; day 5: 17.9 nmol/l±10.0; day 10: 20.9 nmol/l±13.6). Washing of the skin after 10 minutes did not lead to a decreased resorption of the testosterone gel.

Accordingly, washing the skin 10 minutes after application does not influence the pharmacokinetic profile and thereby more effectively diminishes the possibility of transfer of testosterone to third persons.

1.-16. (canceled)

17. A gel composition on alcoholic basis, consisting of 0.01 to 10.0 weight % of at least one steroid, 0.01 to 20.0 weight % of at least one C₃-C₄ diol, 30 to 90 weight % of at least one alcohol, 0.01 to 1 weight % of at least one pH-stabilizer, 0.1 to 6 weight % of at least one poly(meth)acrylic acid-based polymer, optional excipients selected from the group consisting of preservatives, anti-oxidants, stabilizers, solution enhancers, vitamins, coloring agents, and odor-improving agents, and water.

18. The gel composition in accordance with claim 17, wherein the C₃-C₄ diol is propylene glycol.

19. The gel composition in accordance with claim 17, wherein the alcohol is selected from the group consisting of C₂-C₇ alcohols and mixtures thereof.

20. The gel composition in accordance with claim 19, wherein the alcohol is ethanol or propanol.

21. The gel composition in accordance with claim 17, wherein the at least one pH-stabilizer is tromethanol.

22. The gel composition in accordance with claim 17, wherein the steroid content is from 2 to 7 weight %, based on the composition.

23. A method for the treatment and/or prophylaxis of hypogonadism in a male patient, which method comprises administering a gel composition transscrotally to the male patient, wherein the gel composition on alcoholic basis consists of 0.01 to 10.0 weight % of at least one steroid, 0.01 to 20.0 weight % of at least one C₃-C₄ diol, 30 to 90 weight % of at least one alcohol, 0.01 to 1 weight % of at least one pH-stabilizer, 0.1 to 6 weight % of at least one poly(meth)acrylic acid-based polymer, optional excipients selected from the group consisting of preservatives, anti-oxidants, stabilizers, solution enhancers, vitamins, coloring agents, and odor-improving agents, and water, whereupon the male patient is treated for hypogonadism.

24. The method in accordance with claim 23, wherein the androgenic steroid is selected from the group consisting of testosterone, testosterone esters, methyl testosterone, dihydrotestosterone, and mixtures thereof.

25. The method in accordance with claim 23, wherein the steroid content is from 2 to 7 weight %, based on the composition.

26. The method in accordance with claim 23, wherein the composition is administered in an amount from 0.2 to 20.0 grams.

27. The method in accordance with claim 26, wherein the composition is administered in an amount from 1.0 to 7 grams.

28. The gel composition of claim 20, wherein the propanol is isopropanol.

29. A method of treating a disease associated with testosterone shortage in a patient due to hypogonadism, which method comprises topically administering a gel composition comprising at least one androgenic steroid to the patient, wherein the gel composition on alcoholic basis consists of 0.01 to 10.0 weight % of at least one steroid, 0.01 to 20.0 weight % of at least one C₃-C₄ diol, 30 to 90 weight % of at least one alcohol, 0.01 to 1 weight % of at least one pH-stabilizer, 0.1 to 6 weight % of at least one poly(meth)acrylic acid-based polymer, and optional excipients selected from preservatives, anti-oxidants, stabilizers, solution enhancers, vitamins, coloring agents, and odor-improving agents, and water, whereupon the patient is treated therapeutically or prophylactically for a disease associated with testosterone shortage.

30. The method of claim 29, wherein the disease is osteoporosis, muscle atrophy, senescence outfall, loss of libido, impotency, depression or anaemia.

31. The method of claim 29, wherein the gel composition is topically administered to the stomach, arm, leg, breast, back or scrotum.

32. The method of claim 29, wherein the C₃-C₄ diol of the gel composition is propylene glycol.

33. The method of claim 32, wherein the alcohol of the gel composition is selected from the group consisting of C₂-C₇ alcohols and mixtures thereof.

34. The method of claim 33, wherein the alcohol is ethanol or propanol.

35. The method of claim 34, wherein the propanol is isopropanol.

36. The method of claim 29, wherein the pH-stabilizer of the gel composition is tromethamol.

37. The method of claim 29, wherein the steroid is present in an amount from 2 to 7 weight % on alcoholic basis.
